(19) United States
(12) Patent Application Publication
(10) Pub. No.: US 2025/0288754 A1
(43) Pub. Date: Sep. 18, 2025

(54) MINI OVEN AND PERSONAL PYROLYSIZER USING SAME

(71) Applicant: Michael McNeil, Bloomington, IN (US)

(72) Inventor: Michael McNeil, Bloomington, IN (US)

(21) Appl. No.: 19/225,193

(22) Filed: Jun. 2, 2025

Related U.S. Application Data
(63) Continuation-in-part of application No. 18/311,544, filed on May 3, 2023.
Application No. 18/311,544 is a continuation-in-part of application No. PCT/US2020/067275, filed on Dec. 29, 2020.
(60) Provisional application No. 63/348,134, filed on Jun. 2, 2022.
(60) Provisional application No. 63/657,295, filed on Jun. 7, 2024.

(51) Int. Cl. A61M 11/04; A61M 15/00

(52) U.S. Cl. CPC A61M 11/048; A61M 15/0001

(57) ABSTRACT

A method of extracting essential oil from plant matter for personal use includes positioning an amount of medicinal herb in a mini oven. A heating element is at least partially covered by a metallic heat exchanger and positioned in the mini oven. The medicinal herb is pyrolysized by energizing the heating element. Vaporized essential oil from the medicinal herb is moved out of the mini oven and toward the user. Oxygen is displaced from the mini oven responsive to decarboxylation of the medicinal herb, to inhibit combustion at elevated temperatures.

### TECHNICAL FIELD

The present disclosure relates generally to mini ovens, and more particularly to the extraction of essential oil from plant matter with a personal pyrolysizer.

### BACKGROUND

Plants have been the basis for medical treatments throughout much of human history, and such traditional medicine is still widely practiced today. In many instances, the therapeutic compound of interest may be found in the plant's essential oil. For instance, cannabidiol (CBD) has been recognized for its ability to treat stress, anxiety and pain. CBD has been found in high concentrations in the flowers of female hemp plants. There are currently many strategies in practice for separating CBD from plant material and getting the CBD in a usable form to a patient, but all suffer from one drawback or another. Among those drawbacks are cost, excessive processing, loss of ensemble or entourage effect, and a need to add chemicals to a processed product.

The present disclosure is directed toward one or more of the problems set forth above.

### SUMMARY OF THE DISCLOSURE

In one aspect, a method of separating essential oil from a medicinal herb includes opening a mini oven and positioning an amount of the medicinal herb in the mini oven. The mini oven is closed, and the medicinal herb is pyrolysized in the mini oven, which includes displacing oxygen (and other parts of air) from the mini oven with carbon dioxide responsive to decarboxylation of the medicinal herb. Gasified essential oil is moved out of the mini oven through an outlet orifice, whereupon the gasified essential oil condenses into an aerosol cloud. The aerosol cloud of condensed essential oil is moved to a mouth piece. The mini oven is prepared for a subsequent use by removing residue of the medicinal herb, which is mostly biochar, from the mini

In another aspect, a personal essential oil extractor includes a container that defines a condensation chamber. A mini oven includes a heating element positioned within an enclosure. The heating element and the enclosure define a pyrolysizing cooking space that is fluidly connected to the condensation chamber exclusively through at least one outlet orifice. An extraction conduit has one end opening into the condensation chamber and an opposite end that opens through a mouth piece. Gasified compounds generated responsive to pyrolysization in the pyrolysizing cooking space move through the outlet orifice, into the condensation chamber toward the extraction conduit responsive to suction at the mouthpiece. Oxygen in the pyrolysizing cooking space is displaced by carbon dioxide responsive to pyrolysis of medicinal herb positioned in the pyrolysizing cooking space.

In still another aspect, a personal medicinal herb consumption device includes means for pyrolysizing a personal consumption quantity of a medicinal herb with a mini oven. Means, which includes an enclosure of the mini oven, is provided for displacing oxygen from the mini oven with carbon dioxide responsive to decarboxylation of the medicinal herb. Means, which includes an outlet defined by the mini oven, is provided for moving a gasified portion of the medicinal herb generated responsive to the pyrolysization out of the mini oven to a mouth piece. Means, including a size of the outlet, is included for trapping biochar, which is a majority of a residue from the medicinal herb generated responsive to the pyrolysization, in the mini oven.

### BRIEF DESCRIPTION OF THE DRAWINGS

FIG. 1 is a perspective view of a mini oven heating element according to the present disclosure;

FIG. 2 is a front elevational view of the mini oven heating element of FIG. 1;

FIG. 3 is a top view of the heating element of FIG. 2;

FIG. 4 is a bottom view of the heating element;

FIG. 5 is a right side view of the heating element;

FIG. 6 is a front sectioned view of the heating element as viewed along section lines 6-6 of FIG. 3;

FIG. 7 is a sectioned view of the heating element as viewed along section lines 7-7 of FIG. 2;

FIG. 8 is a front sectioned view of a mini oven according to the present disclosure;

FIG. 9 is a partial sectioned front view of a personal essential oil extractor according to the present disclosure;

FIG. 10 is a front elevational view of a personal essential oil extractor according to the present disclosure;

FIG. 11 is a schematic view of an essential oil extractor;

FIG. 12 is a schematic view of another essential oil extractor in association with the mini oven of FIG. 8;

FIG. 13 is a side sectioned view of a personal essential oil extractor according to another embodiment of the present disclosure;

FIG. 14 is an exploded sectioned view of the essential oil extractor of FIG. 13.

FIG. 15 is a side view of a personal essential oil extractor according to still another embodiment of the present disclosure;

FIG. 16 is a side view of a mini oven from the essential oil extractor of FIG. 15;

FIG. 17 is a side exploded view of the mini oven of FIG. 16;

FIG. 18 is a side view of a personal essential oil extractor according to another embodiment of the present disclosure; and

FIG. 19 is a sectioned side view of a portion of the personal essential oil extractor of FIG. 18.

### DETAILED DESCRIPTION

This disclosure teaches a method and apparatus to pyrolysize a small amount of plant matter to separate the plant's essential oil from plant solids, and to instantly deliver that essential oil to a user without intervening processing. As used in the present disclosure, pyrolysis means a process by which plant matter, whether fresh or dried, is irreversibly decomposed at elevated temperatures in an oxygen starved environment. The elevated temperatures can be, and often are, above temperatures at which the plant material would naturally combust in air. This is accomplished by positioning plant matter in contact with a heating element, and positioning both within a mini oven while drawing volatile compounds from the mini oven and limiting a flow of air into the mini oven. After exiting the mini oven, the gasified essential oil quickly condenses as its temperature drops to form an aerosol cloud, which may be visible by a user before being drawn into a user's lungs through a mouth piece. By adjusting a heating element temperature and/or a suction rate at the mouth piece responsive to aerosol cloud density in a window space, a user can efficiently extract and consume essential oil directly from plant material without ingesting significant amounts of harmful smoke particulates and other products of combustion. Pyrolysis of the plant matter leaves behind biochar in the device for disposal after the process is complete.

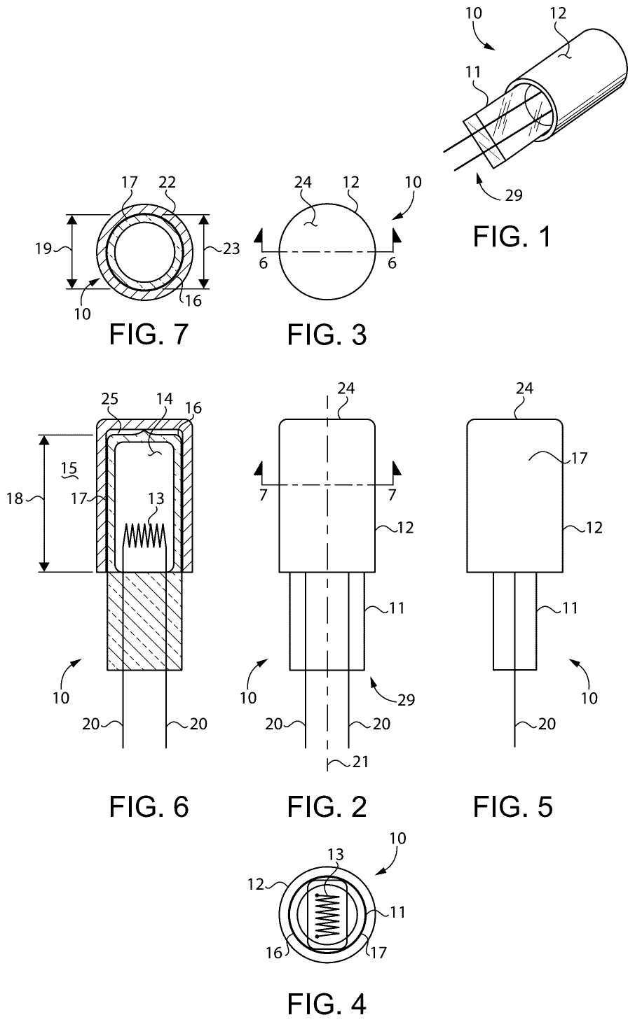

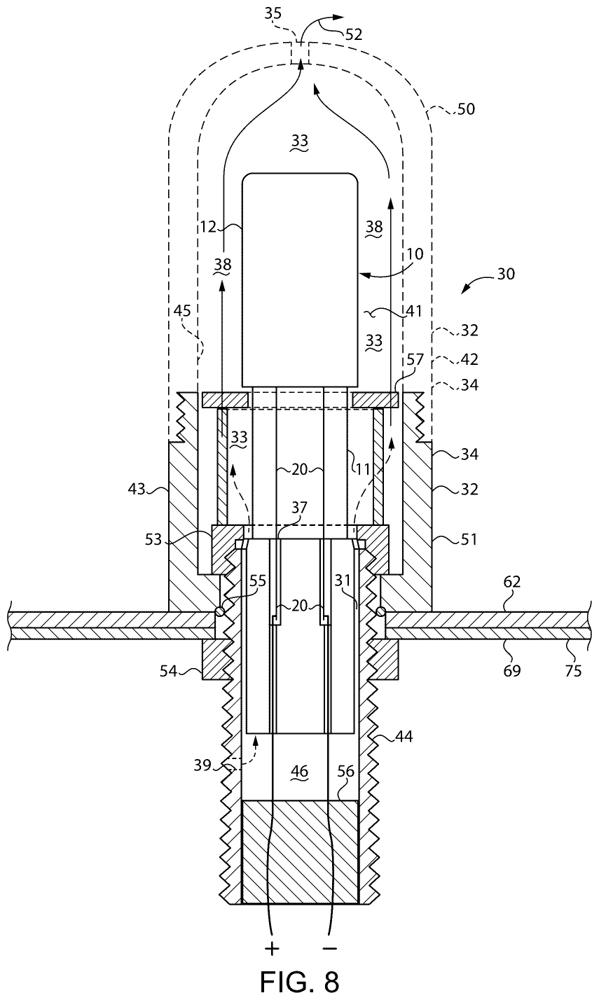

Referring now to FIGS. 1-7, a mini oven heating element 10 according to the present disclosure may include an incandescent lamp bulb 11 and a metallic heat exchanger 12. Incandescent lamp bulb 11 may include a tungsten coil 13 immersed in a gas 14 that is different from air 15 and enclosed in a glass globe 16 that defines a cylindrical segment 17 with a length 18 about equal to a diameter 19 of the cylindrical segment 17. As used in the present disclosure, the phrase “about equal” means that when a ratio of the lesser to the greater of the two dimensions is rounded to a single integer, that number is one. Thus, in this case, the ratio of the diameter 19 to length 18, or possibly vice versa, when rounded to a single integer is the integer one. The incandescent lamp bulb 11 may also include two electrical conductors 20 extending parallel to a centerline 21 of the cylindrical segment 17 and sized to mate to a lamp socket base shown infra. The metallic heat exchanger 12 is in contact with, and at least partially covers, the cylindrical segment 17 of the glass globe 16. In general, the purpose of the metallic heat exchanger 12 is to assist in converting all of the electromagnetic emissions from gas 14 and coil 13 into heat. Furthermore, by choosing a material with good heat conduction, the temperature at the outer surface of the metallic heat exchanger 12 may be somewhat uniform even though temperatures at different locations on glass globe 16 may vary due to, among other things, proximity to tungsten coil 13. The metallic heat exchanger 12 is preferably mechanically joined to glass globe 16, such as by a friction fit, in order to reduce the likelihood of undesirable generation of harmful gases that could originate from adhesives and the like. Nevertheless, metallic heat exchanger 12 can be joined to incandescent lamp bulb 11 in any manner known in the art without departing from the present disclosure. At a minimum, the connection between the metallic heat exchanger 12 and the incandescent lamp bulb 11 is sufficiently strong that the heating element 11 can be inverted without the metallic heat exchanger 12 falling off of glass globe 16.

Metallic heat exchanger 12 may include a copper or aluminum tube 22 with an inner diameter 23 that matches the diameter of cylindrical segment 17. Those skilled in the art will appreciate that copper and aluminum are identified because of their relatively low cost and high thermal conductivity, with copper being preferred. Other metals or alloys could be used without departing from the present disclosure. Metallic heat exchanger 12 might also include a end cover 24 that covers most to all of end 25 of glass globe 16. In one specific example, in the event that the metallic heat exchanger 12 is a segment of copper or aluminum tube 22, end cover 24 might be formed by bending over the top edges of the tube to mostly cover the end 25 of glass globe 16. The copper or aluminum tube 22 may have a thickness about equal to one millimeter, but other thicknesses might also work sufficiently well. For instance, metallic heat exchanger 12 could include one or more layers of aluminum foil. Preferably, the wall thickness of copper or aluminum tube 22 is about equal to the wall thickness of glass globe 16. Preferably, the metallic heat exchanger 12 has a greater thermal mass and a higher thermal conductivity than that of glass globe 16. In one specific example, incandescent lamp bulb 11 could be a 12 Volt halogen lamp bulb 8 with a G4 base configuration 29.

Referring now in addition to FIG. 8, a mini oven 30 includes a mini oven heating element 10 that includes an incandescent lamp bulb 11 in contact with, and at least partially covered by, a heat exchanger 12. The incandescent lamp bulb 11 is received in a counterpart matching lamp socket base 31. Mini oven 30 includes an enclosure 32 that defines an interior space 33, includes an external surface 34, and defines at least one outlet orifice 35. The mini oven heating element 10 is positioned within the interior space 33 and is electrically connected to the lamp socket base 31. At least one of the enclosure 32 and the lamp socket base 31 may define at least one inlet orifice 37, if included. The mini oven heating element 10 and the enclosure 32 define a pyrolysizing cooking space 38. The pyrolysizing cooking space 38 may have a total volume that is less than 10 ml. The interior space 33, which includes pyrolysizing cooking space 38, is fluidly isolated from the external surface 34 except through outlet orifice 35 and inlet orifice 37, if included. A “pyrolysizing cooking space” means a space in which pyrolysis of medicinal herb is capable of occurring within a normal range of operation of a heating element.

In one example embodiment, a cylindrical container 42 and detachable cover 43 are threadably connected, and may or may not include an o-ring seal at the threaded connection. In this embodiment, cylindrical container 42 and detachable cover 43 are inverted with the cover 43 on the bottom and the container 42 on top. The lamp socket base 31 may be mounted to the cover 43, and the cylindrical container 42 may define the outlet orifice 35. In the configuration shown, the inlet orifice 37 may extend through the detachable cover 43. The term detachable is used merely to illustrate that the enclosure preferably includes at least two components that may be opened by being detached from one another, such as for loading plant material therein. The lamp socket base 31 may be mounted in an externally threaded lamp tube 44 that extends through, and is itself mounted to, the detachable cover 43. The lamp socket base 31 may be fixed in place by pinching its end flange between one end of the externally threaded lamp tube 44 and a cap nut 53. The externally threaded lamp tube 44 may itself be mounted to detachable cover 43 with the use of a nut 54 that clamps an radially inwardly directed flange portion of detachable cover 43 between nut 54 and cap nut 53. An o-ring seal 55 in contact with lamp tube 44 and container 42 might be included. The externally threaded lamp tube 44 may extend through a bore defined through one end of detachable cover 43.

In the illustrated embodiment, enclosure 32 is illustrated as an aluminum capsule 50 with a metallic threadably attached cover 51, and a volume of likely less than 10 ml. The inlet orifice 37 may extend into the externally threaded lamp tube 44 through the lamp socket openings or around the lamp socket base 31. In an alternative embodiment, detachable cover 43 could be a threadedly attachable metallic lid for a small glass jar, which would comprise the cylindrical container 42. Preferably, the enclosure 32 includes an aluminum reflection surface 45 facing the metallic heat exchanger 12. The reflection surface 45 reflects infrared rays back into the pyrolysizing cooking space 38. This may be accomplished by utilizing the aluminum capsule 50 shown, or might be accomplished, for instance, by wrapping aluminum foil on the exterior surface of a miniature glass jar, if such is used for the enclosure 32. Other detachable connections besides threaded connections could be substituted without departing from the present disclosure. For instance, a mere friction fit between the two components that make up enclosure 32 would also fall within the intended scope of the present disclosure. If a threaded connection as shown, one or more pins (not shown) could extend from interior space 33, through the bottom wall of enclosure 32, and through removable lid 62 and maybe into battery container 69, to inhibit relative rotation when threading and unthreading the enclosure 32.

In order to better control the sizing and location of any air flow into enclosure 32, one or more o-rings, such as o-ring 55, may be positioned along the length of threaded lamp tube 44 to inhibit gas or air leakage at one or more of the component contact interfaces. A plug 56, such as food grade high temperature silicon sealant, may be positioned to close one end of threaded lamp tube 34 so that the inside space 46 of tube 44 essentially becomes part of the interior space 33 of enclosure 32. With appropriate sealing strategies, interior space 33 can be a closed volume except by the inclusion of outlet orifice 35 and air inlet orifice 39, if included. In other versions, either by the omission of adequate seals at component contact locations and/or by the inclusion of an air inlet orifice 39 through the wall of lamp tube 44 may be included. In this way, air can flow in through air inlet 39, up through lamp tube 44 and around or through lamp socket 31 and then through inlet orifice 37 of interior space 33 to pyrolysizing cooking space 38. Flow characteristics into, through and out of interior space 33 can further be controlled via selecting relative area sizing for air inlet orifice 39 and outlet orifice 35. In the event that inlet orifice 37 has a smaller flow area than air inlet 39, the ratio of inlet orifice 37 to outlet orifice 35 would be more controlling over flow through interior space 33, recognizing that much of the gas that flows out of outlet orifice 35 originates in interior space 33 rather than entering through an inlet orifice 37, if included. Both inlet orifice 37 and air inlet orifice 39 may be omitted.

In one example embodiment, an internal wall 57 and wall support 58 may be included to further define pyrolysizing cooking space 38 to have a hollow cylindrical shape bound on the inside by the heating element 10 and on the outside by the aluminum reflection surface 45. In this manner, plant material positioned in pyrolysizing cooking space 38 is located in an infrared cross fire to promote rapid and distributed heat transfer. Those skilled in the art will appreciate that if an inlet orifice 37 is included, it can be located in several locations, including through the bottom or side wall of enclosure 32 and elsewhere without departing from the scope of the present disclosure. It might be desirable to size the pyrolysizing cooking space 38 to have a cross sectional area at least one and maybe two or more orders of magnitude greater than a combined total flow area of the at least outlet orifice 35. This strategy both serves to starve the pyrolysizing cooking space 38 of oxygen, but also to trap solids in the pyrolysizing cooking space 38.

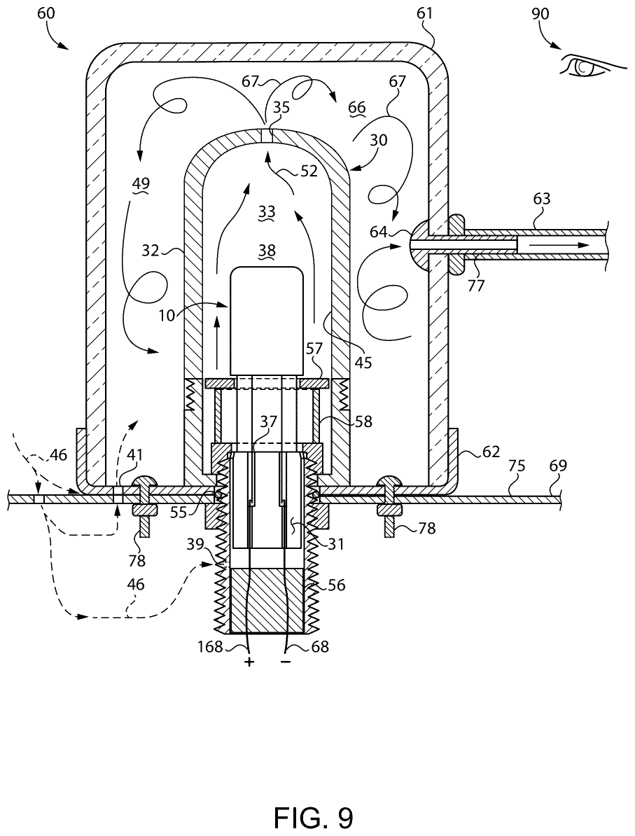

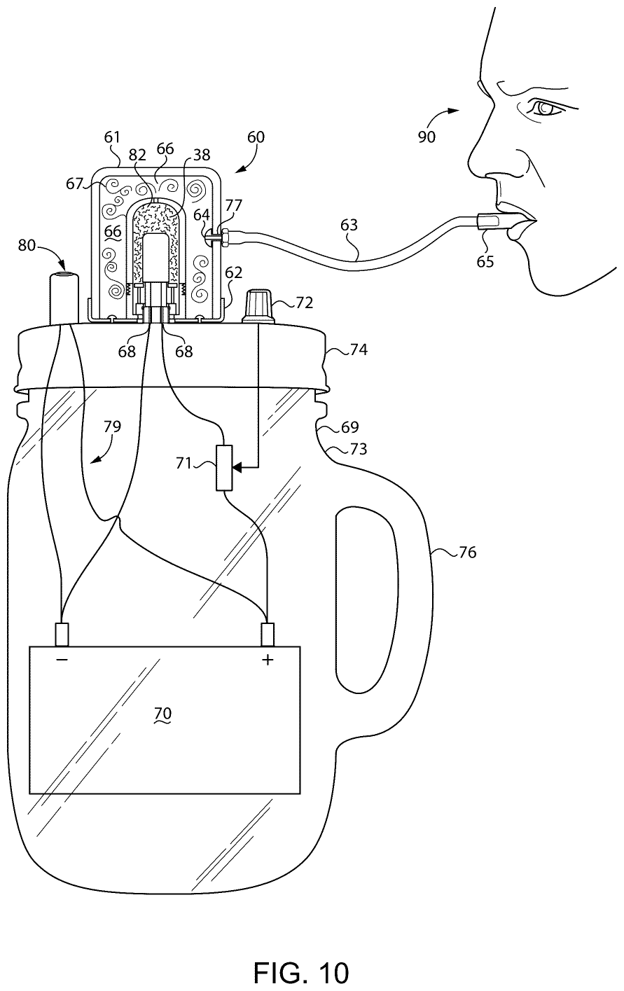

Referring now in addition to FIG. 9, a personal essential oil extractor 60 includes a container 61 that defines a condensation chamber 49. A mini oven 30 includes a heating element 10, which may be the same or different from that described previously, that is positioned within an enclosure 32. The heating element 10 and the enclosure 32 define a pyrolysizing cooking space 38 that is fluidly connected to the condensation chamber 49 exclusively through at least one outlet orifice 35. An extraction conduit 63 has one end 64 that opens into condensation chamber 49, and an opposite end that opens through a mouth piece 65 (FIG. 10). Gasified compounds generated responsive to pyrolysis in the pyrolysizing cooking space 38 move through the outlet orifice 35, into the condensation chamber 49 toward the extraction conduit 63 responsive to suction at the mouth piece 65. Although not necessary, the extraction conduit 63 may be fluidly connected to the window space 66 with a hollow fastener 77 that extends through a side wall of container 61. Alternatively, extraction conduit 63 could be received in the bore of an externally threaded tube mounted through the side wall or top of container 61.

In order to possibly provide a user with visible feedback as to the operation of essential oil extractor 60, the condensation chamber 49 may include a window space 66 that is visible from outside the container 61 through a window, which may be a portion of container 61. The outlet orifice 35 may be fluidly separated from the end 64 of the extraction conduit 63 by the window space 66. With this arrangement, essential oil that is gasified in the mini oven 30 and moved through the outlet orifice 35 condenses to an aerosol cloud visible in the window space 66 through the window by a user 90 of the essential oil extractor 60. In some embodiments, the pyrolysizing cooking space 38, with an exception of the outlet orifice 35, is a closed volume. The illustrated embodiment, however, differs by the inclusion of an inlet orifice 37 that is fluidly connected to air inlet 39 around or through lamp socket base 31. Thus, in the illustrated embodiment, air may travel into and through pyrolysizing cooking space 38. On the otherhand, if inlet orifice 37 is omitted, gasified products generated in pyrolysizing cooking space 38 move to relieve pressure through outlet orifice 35 due to the gasification in the pyrolysizing cooking space 38.

In the illustrated embodiment, the pyrolysizing cooking space 38 and its associated enclosure 32 are positioned inside of container 61. In other embodiments, the pyrolysizing cooking space 38 and some or all of container 32 may be positioned outside of container 61 without departing from the present disclosure. For instance, FIG. 12 shows such an alternative. By including enclosure 32 within container 61 as shown, the window space 66 may inherently serve as an insulating layer between the relatively hot mini oven 30 and the container 61 so that a user 90 is not exposed to any dangerously high temperature surfaces.

The present disclosure also contemplates embodiments in which the condensation chamber 49 of the container 61, with an exception of a fluid connection to the extraction conduit 63 and the outlet orifice 35, is a closed volume. In such an embodiment, suction at the mouth piece 65 evacuates some of the content of window space 66 while lowering pressure in condensation chamber 49, possibly creating a pressure gradient through outlet orifice 35 to promote movement of gasified compounds from pyrolysization cooking space 38 into window space 66. A bypass orifice 41 may open directly into condensation chamber 66 and bypass the pyrolysizing cooking space 38. In this manner, any air drawn through bypass orifice 41 mixes with molecules ejected from pyrolysizing cooking space 38 through outlet orifice 35 toward extraction conduit 63. Although not necessary, the illustrated embodiment may include a wall support 58 and internal wall 57 to inhibit plant matter placed in pyrolysizing cooking space 38 from falling down into the cooler areas of interior space 33 adjacent the base of heating element 10 and lamp socket base 31. Preferably, the enclosure 32 includes an aluminum reflection surface 45 that encircles and faces heating element 10. Other infrared reflective surfaces could be substituted in the place of an aluminum reflective surface without departing from the present disclosure, or omitted altogether. For instance, if enclosure 32 includes a glass cylinder, such as a small glass jar, an aluminum or other infrared reflective surface may be omitted so that an observer 90 could actually see into pyrolysizing cooking space 38 from outside of container 61. In addition to enclosure 32 possibly comprising a small glass jar with a removable lid, the container 61 may comprise a transparent glass jar with a removable lid 62. The removable lid 62 may be connected to the transparent glass jar via conventional threads. In such a case, separate fasteners 78 may extend through removable lid 62 so that lid 62 remains fixed when the glass jar of container 61 is rotated to be detached therefrom in order to access enclosure 32.

Referring now in addition to FIG. 10, essential oil extractor 60 may include a battery container 69 that houses a battery 70 that may be electrically connected to heating element 10. In other embodiments, battery 70 may be eliminated in favor of another power source, such as a wall socket or a USB port. The electrical connection between battery 70 and heating element 10 may include an analog potentiometer 71 with a manual adjustment knob 72 positioned outside of battery container 69. In one specific embodiment, battery container 69 may include a housing 73 that is covered by a threaded ring 74 and a disk 75. The removable lid 62 of container 61 may be attached to disk 75 by fasteners 78 as best shown in FIG. 9. Thus, housing 73 could comprise a canning type jar with a typical lid that includes a threaded ring 74 and disk 75. In this embodiment, housing 73 includes a mug handle 76 to make the essential oil extractor 60 more portable. The essential oil extractor 60 may also include a charging circuit 79 that is electrically connected to battery 70 and includes a charging port 80 exposed outside of battery container 69 so that battery 70 can be electrically connected to a charger when essential oil extractor 60 is not in use.

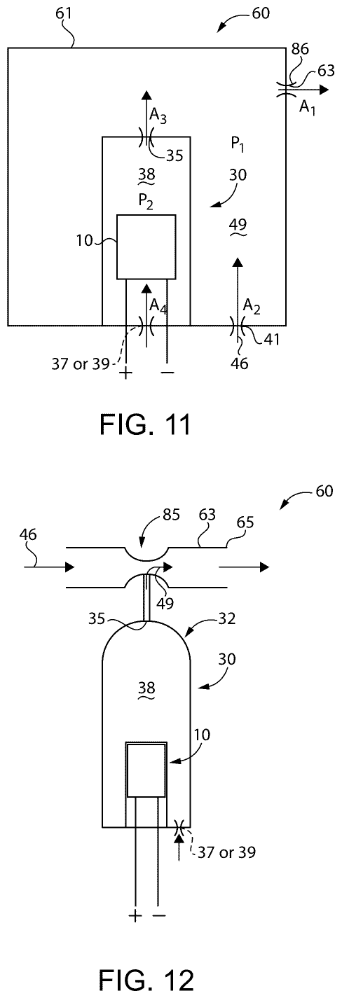

Referring now in addition to FIG. 11, a schematic view of an essential oil extractor 60 is illustrated with the various orifices, some of which may be excluded in various embodiments of the present disclosure. For instance, the essential oil extractor will always include an outlet orifice 35 that fluidly connects the pyrolysizing cooking space 38 to the condensation chamber 49, and a suction orifice 86 that may be located anywhere along extraction conduit 63. In the illustrated embodiment shown earlier, the suction orifice 86 may be considered as being located in the hollow fastener 77. The embodiment shown includes a bypass orifice 41 and an inlet orifice 37, one or both of which may be excluded without departing from the present disclosure. The suction orifice 86 may be thought of as having an area A₁, the bypass orifice 41 may be thought of as having an area A₂, the outlet orifice 35 may be thought of as having a flow area A₃, and the smaller of inlet orifice 37 and air inlet 39 may be thought of as having a flow area of A₄. The pressure in condensation chamber 49 is identified as P₁, and the pressure in pyrolysizing cooking space 38 has been identified as P₂. The table below shows some example area combinations according to the present disclosure.

### EXAMPLE EMBODIMENTS (mm²)

| A₁ | A₂ | A₃ | A₄ |
|---|---|---|---|
| 13 | Ø | 3 | Ø |
| 13 | 3 | 9 | 3 |
| 13 | 6 | 9 | 0.3 |
| 13 | 3 | 9 | 1 |
| 13 | Ø | 9 | 3 |
| 13 | 3 | 9 | Ø |

Those skilled in the art will appreciate that when no suction is occurring in extraction conduit 63, the pressure outside of essential oil extractor 60, the pressure P₁ in condensation chamber 49, and the pressure P₂ in pyrolysizing cooking space 38 may be all at atmospheric pressure. When heating element 10 is active, the pressure P₂ in pyrolysizing cooking space 38 would tend to increase due to the gasified molecules occupying much greater space than those same molecules would in a solid or liquid form prior to the application heat. The pressure P₁ in condensation chamber 49 would tend to drop below atmospheric pressure responsive to suction at orifice 86 generated by a user at the mouthpiece 65 (FIG. 10). In most instances, both A₁ and A₃ will be greater than A₄. Furthermore, in order to promote a pressure drop in condensation chamber 49 responsive to suction at suction orifice 86, the area A₁ will usually be greater than the sum of A₂ plus the lesser of A₃ and A₄. Thus, during the operation of the device when heat is being generated by heating element 10 and suction is being applied at suction orifice 86, the pressure P₁ will be less than P₂. One with ordinary skill in the art can choose various sizing for these areas, if included, in order to limit the supply of oxygen to pyrolysizing cooking space 38 while moving a sufficient flow of gas into extraction conduit 63 at a rate deemed desirable by a user.

Referring in addition to FIG. 12, an essential oil extractor 60 according to another embodiment of the invention positions the enclosure 32 and pyrolysizing cooking space 38 outside of the condensation chamber 49. Instead, in this embodiment, the pyrolysizing cooking space 38 is fluidly connected to the condensation chamber 49 by way of a venturi 85 so that pressure in extraction conduit 63 is at its lowest precisely where the fluid connection occurs in order to promote movement of gasified molecules from pyrolysizing cooking space 38 into condensation chamber 49 on the way to mouth piece 65.

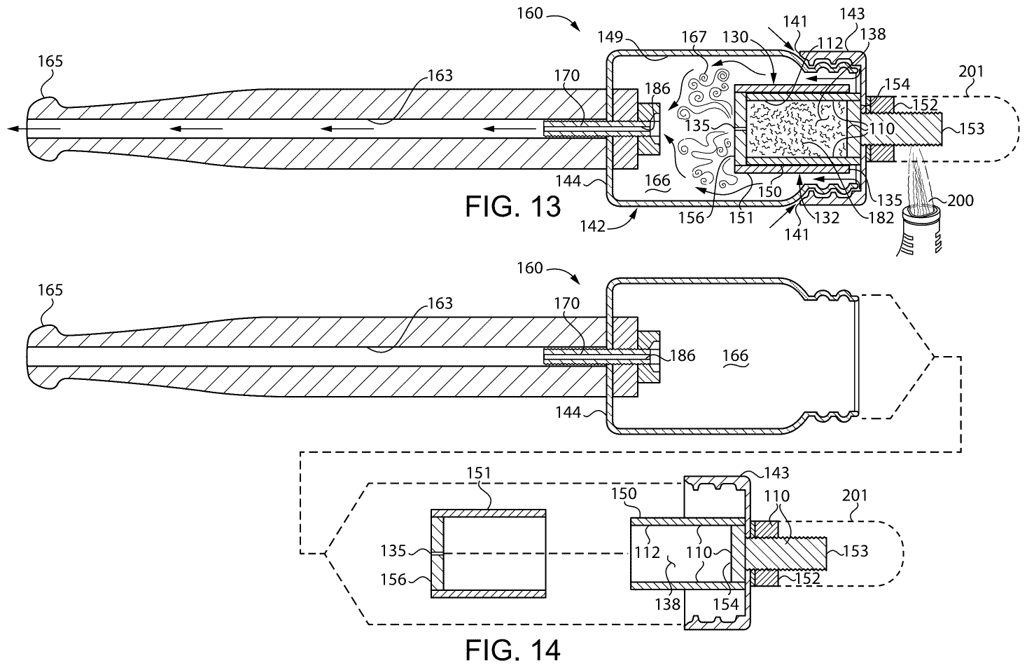

In another embodiment, the heat for pyrolyzation may be provided by a flame as opposed to being electrically generated as in the previous embodiment. In particular, FIGS. 13 and 14 show an essential oil extractor 160 that includes a container 142 that may include a transparent glass jar 144 (e.g., 5-10 ml) with a heat tolerant aluminum cover 143. The clearance between jar 144 and cover 143 defines a bypass orifice 141 with a flow area of A2 according to the previous table. Tightening cover 143 to the jar 144 drives the flow area A2 towards zero, and allows a user to adjust the area A2 by stopping cover 143 short of full tight on jar 144. A heating element 110 also functions as an enclosure 132 of mini oven 130 that defines pyrolysizing cooking space 138, which may have a small volume, maybe less than 1 ml. Thus, heating element 110 can be considered to be positioned in enclosure 132, and together the heating element 110 and enclosure 132 define pyrolysizing cooking space 138. As used in this disclosure, the phrase “a mini oven including a heating element positioned within an enclosure” means that a surface of the heating element defines a portion, or all of, the pyrolysizing cooking space 38, 138. Enclosure 132 may comprise telescoping copper tubes, with a first tube 150 attached in press fit contact with a head 154 of a threaded copper fastener 153 that extends through cover 143, and a second tube 151 at least partially closed at one end with maybe a press fit copper disc 156, which may define an outlet orifice 135 (area A3 from previous table). Alternatively, copper disc 156 may not define a hole, leaving the clearance between tubes 150 and 151 to constitute the outlet orifice 135 (area A3 from previous table) that fluidly connects the pyrolysizing cooking space 138 to a condensation chamber 149 defined by container 142. The area A3 equals a sum of all the outlet orifices 135, if more than one is included. A copper nut 152 may be threaded to fastener 153 to securely attach first tube 150 to an inner surface of cover 143. Copper is suggested for its superior heat conducting properties, but other metals (e.g., silver, aluminum, steel, titanium, et al.) and their alloys (e.g., brass, stainless steel, etc.) could also be utilized without departing from this disclosure. The copper functions as a good heat exchanger to transfer heat from a heat source, such as flame 200, applied to the exposed fastener 153 and nut 152 to transfer heat to a personal quantity of medicinal herb 182 positioned in pyrolysizing cooking space 138. The copper components preferably have good contact area with each other to better facilitate heat transfer throughout mini oven 130. Instead of a flame 200, the red hot surface of a car cigarette lighter or other heat source could be utilized. A ferromagnetic fastener 201 (or other suitable metallic piece) may be attached to cover the exposed end of copper fastener 153. Heating may be accomplished by inserting fastener 201 into an induction heating coil (not shown) as an alternative to flame 200 to heat heating element 110.

A bottom end of glass jar 144 may define a hole therethrough for attachment to one end of an extraction conduit 163, such as by a vented screw 170 that defines a suction orifice 186 with a flow area A1 according to the previous table. Preferably A1 is greater than A2 so that a vacuum (relative to ambient pressure) is generated in condensation chamber 149 responsive to suction at a mouthpiece 165 that is fluidly connected to an opposite end of extraction conduit 163. A steady stream of cooling bypass air (see arrows in FIG. 13) is drawn through bypass orifice 141, through condensation chamber 149, past mini oven 130 and into extraction conduit 163 to mouthpiece 165 responsive to suction at mouthpiece 165 by a user. Gasses generated in pyrolysizing cooking space 138 responsive to flame heat move through outlet orifice 135 into condensation chamber 149, where they condense into an aerosol cloud 167 that accumulates in condensation chamber 149 and is carried toward the mouthpiece 165 by the stream of bypass air. A user may adjust one or both of suction rate and flame heat rate independently responsive to a density of the aerosol cloud 167 visible in window space 166. No air, bypass or ambient, is drawn into mini oven 130 responsive to suction at the mouthpiece 165 because, in this embodiment, except for outlet orifice(s) 135, pyrolysizing cooking space 138 is a closed volume. A positive pressure gradient from the heated pyrolysizing cooking space 138 through outlet orifice 135 prevents any of the bypass air from entering the mini oven 130 from the condensation chamber 149. The pressure gradient is mostly attributable to decarboxylation and gasification of molecules in the herb 182. Furthermore, in the context of the previous table, in this embodiment mini oven 130 does not include any separate fluid connection to ambient air, rendering the flow area A4 (see previous table) as zero for the essential oil extractor 160.

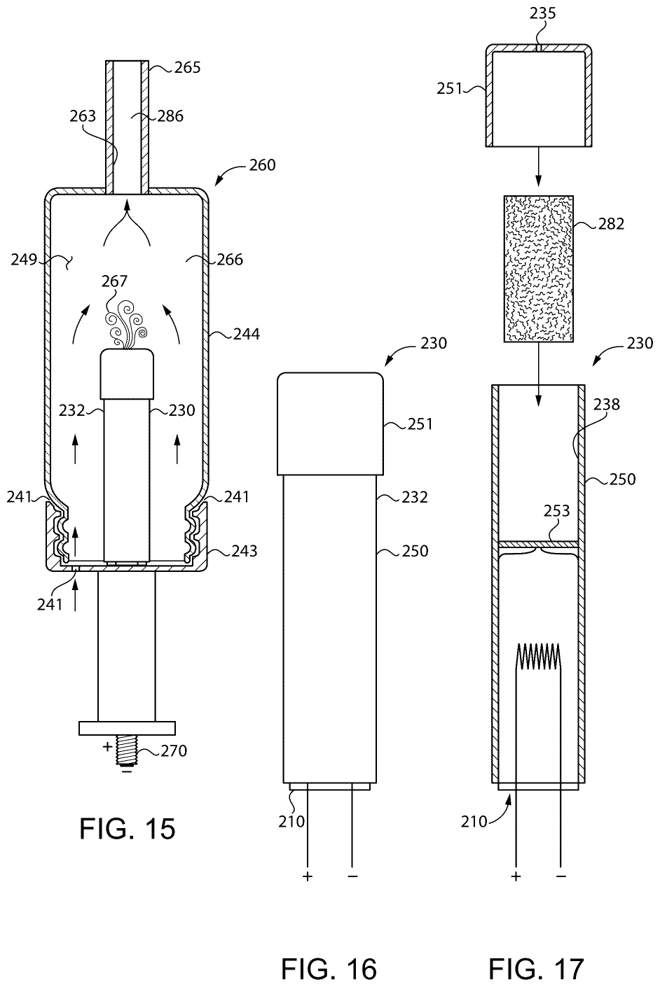

In still another embodiment, the heat for pyrolyzation may again be electrically generated as in the earlier described embodiment. In particular, FIGS. 15-17 show an essential oil extractor 260 that includes a container 242 that may include a transparent glass jar 244 (e.g., 10-15 ml) with a heat tolerant aluminum cover 243. The clearance between jar 244 and cover 243 may define a bypass orifice 241 with a flow area of A2 according to the previous table. Tightening cover 243 to the jar 244 drives the flow area A2 towards zero, and allows a user to adjust the area A2 by stopping cover 143 short of full tight on jar 144. Alternatively, or in addition to the cover/jar clearance, bypass orifice 241 (area A2) may be provided via an orifice defined by cover 243, as shown, or located elsewhere (e.g., through glass jar 244. A heating element 210 may take the form of an electric coil that may be part of an incandescent light bulb, such as a halogen light bulb. Heating element 210 may also function as a portion of an enclosure 232 of mini oven 230 that defines pyrolysizing cooking space 238, which may have a small volume, maybe less than 1 ml. Heating element 210 can be considered to be positioned in enclosure 232, and together the heating element 210 and enclosure 232 define pyrolysizing cooking space 238. As used in this disclosure, the phrase “a mini oven including a heating element positioned within an enclosure” means that a surface of the heating element defines a portion, or all of, the pyrolysizing cooking space 38, 138, 238. Enclosure 232 may comprise telescoping copper piping, with a copper tube 250 slidably received in a copper end cap 251. The heating element may be press fit into one end of copper tube 250, and a separator 253 may separate the heating element 210 from the pyrolysizing cooking space 238. Copper end cap 251 may define an outlet orifice 235 (area A3 from previous table). Alternatively, or in addition to the outlet orifice 235 defined by copper end cap 251, the clearance between copper tube 250 and copper end cap 251 may comprise all or portion of outlet orifice 235 and hence a portion or all of area A3 from the previous table. The outlet orifice 235 (area A3 from previous table) fluidly connects the pyrolysizing cooking space 238 to a condensation chamber 249 defined by container 242. The area A3 equals a sum of all the outlet orifices 235, if more than one is included. The copper functions as a good heat exchanger to transfer heat from a heat source, such as heating element 210, applied to the copper tube 250 to transfer heat to a personal quantity of medicinal herb 282, which may be a packed cylinder, positioned in pyrolysizing cooking space 238. The copper components preferably have good contact area with each other to better facilitate heat transfer throughout mini oven 230.

A bottom end of glass jar 244 may define a hole therethrough for attachment to one end of an extraction conduit 263, that defines a suction orifice 286 with a flow area A1 according to the previous table. Preferably A1 is greater than A2 so that a vacuum (relative to ambient pressure) is generated in condensation chamber 249 responsive to suction at a mouthpiece 265 that is fluidly connected to an opposite end of extraction conduit 263. A steady stream of cooling bypass air (see arrows in FIG. 15) is drawn through bypass orifice 241, through condensation chamber 249, past mini oven 230 and into extraction conduit 263 to mouthpiece 265 responsive to suction at mouthpiece 265 by a user. Gasses generated in pyrolysizing cooking space 238 responsive to heat move through outlet orifice 235 into condensation chamber 249, where they condense into an aerosol cloud 267 that accumulates in condensation chamber 249 and is carried toward the mouthpiece 265 by the stream of bypass air. A user may adjust one or both of suction rate and heat rate independently responsive to a density of the aerosol cloud 267 visible in window space 266. No air, bypass or ambient, is drawn into mini oven 230 responsive to suction at the mouthpiece 265 because, in this embodiment, except for outlet orifice(s) 235, pyrolysizing cooking space 238 is a closed volume. A clearance between heating element 210 and copper tube 250 may comprise all or part of outlet orifice 235 without departing from this disclosure. A positive pressure gradient from the heated pyrolysizing cooking space 238 through outlet orifice 235 prevents any of the bypass air from entering the mini oven 230 from the condensation chamber 249. The pressure gradient is mostly attributable to decarboxylation and gasification of molecules, including water moisture, in the herb 282. Furthermore, in the context of the previous table, in this embodiment mini oven 230 does not include any separate fluid connection to ambient air, rendering the flow area A4 (see previous table) as zero for the essential oil extractor 260. Essential oil extractor 260 may be connected to a battery or other electric power source (not shown) via a threaded battery attachment 270, which may comprise a standard 510 thread pattern.

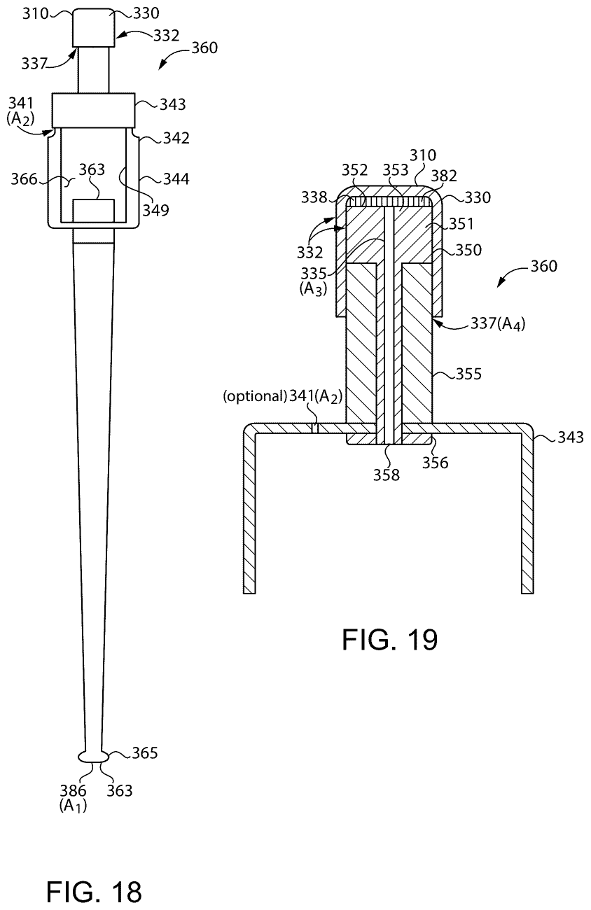

Referring now to FIGS. 18 and 19, a personal essential oil extractor 360 is similar to the embodiment of FIGS. 13 and 14, except the mini oven 330 and its associated pyrolysizing cooking space 338 are positioned outside the container 342 that defines the condensation chamber 349. Like the earlier embodiment, container 342 may include a transparent glass jar 344 with a heat tolerant threaded aluminum cover 343. Any clearance between cover 343 and jar 344 defines a bypass orifice 341, which corresponds to flow area A2 from the previous table. In this example, cover 343 may be tight on jar 344 so that the bypass orifice 341 is zero. As shown in FIG. 19, an optional orifice 341 may be defined through lid 343 to allow some predefined bypass flow area A2. A vented screw 350 is attached to extend through cover 343 by a long nut 355 (e.g., cylindrical threaded spacer) and a nut 356. A head 351 of the vented screw 350 has a cylindrical shape with an exterior diameter that closely matches an interior diameter of a heating element 310, which may comprise a copper end cap slide fitting 332. Alternatively, the vented screw 350 could be inverted with its head 351 positioned inside the cover 343, and secured in place with a threaded spacer with a cylindrical outer diameter that matches for a slide fit with a copper end cap slide fitting 232 (heating element 310). Heating element 310 may have a sliding friction fit with the head 351, but alternatives such as threaded attachment could be utilized. The head 351 defines a hex tool opening 353 that is surrounded by a top annular surface 352. When mated together, heating element 310 and head 351 are an enclosure 332 that defines a mini oven 330 that includes the pyrolysizing cooking space 338 shown holding a personal quantity of medicinal herb 382, which may be compressed into a thin disk that may be about 1 mm thick and a diameter of about 10 mm, when rounding to the nearest millimeter, so that the herb 382 is smashed to occupy between fifty and one hundred cubic millimeters, for example The clearance between an interior surface of heating element 310 and head 351 and long nut 355 may define an inlet orifice 337 that may have a zero flow area (A4 from table). The flow area A4 may be zero due to the close fit between heating element 310 and head 351 as well as the compressed herb 382 acting as a gasket blocking any substantial air flow into the pyrolysizing cooking space 338, even in the presence of suction at mouthpiece 365. Heating element 310 may be energized by the application of a flame to its outer surface. This embodiment may be close to the first or last line of the table above, with the outlet orifice 335 (area A3 from table) from the mini oven 310 being defined by one end of a vent hole defined by the vented screw 350, and a second end 358 that opens into a condensation chamber 349. Like the earlier embodiments, the condensation chamber 349 is fluidly connected to the mouthpiece 365 via an extraction conduit 363 that defines a suction orifice 386 (Area A1 from table), which may be located anywhere along its length.

The essential oil extractor(s) 60, 160, 260, 360 taught in this disclosure can also be considered as being a personal medicinal herb consumption device that includes a means for pyrolysizing a personal consumption quantity of a medicinal herb. This means always includes a mini oven as described previously. The device also includes means for displacing oxygen from an enclosure of the mini oven with carbon dioxide responsive to decarboxylation of the medicinal herb. The enclosure limits or eliminates inflow of air, even when suction at the mouthpiece occurs. In addition, the device includes a means for moving a gasified portion of the medicinal herb generated responsive to the pyrolysization out of the mini oven to a mouth piece. This means includes an outlet defined by the mini oven. Finally, the device includes a means for trapping biochar in the mini oven. This means includes a size of the outlet orifice. In all versions of the present disclosure, biochar is a majority of a residue from the medicinal herb generated responsive to the pyrolysization.

### INDUSTRIAL APPLICABILITY

A method of separating essential oil from a medicinal herb includes a step of opening a mini oven 30, 130, 230, 330. In the first illustrated embodiment, the mini oven 30 may be opened such as by unthreading top portion 52 from cover 51. Those skilled in the art will appreciate that mini oven 30 will typically include two or more components that may be separated from one another in order to facilitate opening of the mini oven 30 for loading and unloading. In the case of essential oil extractor 160, cover 143 is detached from jar 144, and second tube 151 is moved out of contact with first tube 150 to open mini oven 130, as shown in the exploded view of FIG. 14. Then, a personal quantity (e.g., 0.5 ml packed) medicinal herb 182 may be packed into first tube 150 to occupy a majority of the volume therein. Even if first tube is tightly packed, generated gasses move easily through outlet orifice 135 and out of pyrolysizing cooking space 138. In a similar manner essential oil extractor 260 may be opened by unscrewing jar 244 from cover 243 and then sliding copper end cap 251 off of copper tube 250 as shown in FIG. 17. In the case of extractor 360, mini oven 330 is opened by sliding heating element 310 out of contact with head 351. Next, a personal amount of medicinal herb 82,182, 282, 382 (e.g., 0.1≤1.0 gm) is positioned in the mini oven 30, 130, 230, 330, which is then closed. In the case of extractor 330, the herb may be smashed into a thin disk in contact with an interior surface of heating element 310 with a separate tool, such as the end of a rod that is not shown so that heat is quickly transferred through the thickness (1 mm) of copper end cap 332 to the herb 382, which has a large contact area with an interior surface of end cap 332. Preferably, the volume of the compressed herb 82, 182, 282, 382 is a majority, and maybe about equal to, a volume of the pyrolysizing cooking space 38, 138, 238, 338. About equal means that a ratio of the two volumes, when rounded to a single digit is one (1). The medicinal herb 82, 182, 282, 382 is then pyrolysized in the mini oven 30, 130, 230, 330 by generating heat with heating element 10, 110, 210, 310 while starving the pyrolysizing cooking space 38, 138, 238, 338 of oxygen, which is accomplished by limiting or eliminating any flow of air into pyrolysizing cooking space 38, 138, 238, 338. Those skilled in the art will appreciate that oxygen may be further limited in pyrolysizing cooking space 38, 138, 238, 338 when certain molecules in the essential oil of the plant matter 82, 182, 282, 382 decarboxylate to produce carbon dioxide responsive to heat. Oxygen may also be displaced from mini oven 30, 130, 230, 330 when water moisture, if any, in the plant matter 82, 182, 282, 382 becomes steam. Thus, one could expect the carbon dioxide generated from decarboxylation and steam from heat to displace oxygen from pyrolysizing cooking space 38, 138, 238, 338, creating an oxygen starved environment. Decarboxylation is a part of pyrolysis, even when suction at the mouthpiece is occuring. This carbon dioxide can displace and/or dilute the amount of oxygen present in pyrolysizing cooking space 38, 138, 238, 338. Decarboxylation may mostly be complete before oil gasification begins, or continue ongoing as gasification occurs. Combustion, even at elevated temperatures, cannot occur in a CO₂ rich environment. Gasified essential oil is moved out of the mini oven 30, 130, 230, 330 through the outlet orifice 35, 135, 235, 335. This movement may be accomplished by creating a pressure differential at outlet orifice 35, 135, 235, 335 by a combination of the generation of gasified molecules in pyrolysization cooking space 38, 138, 238, 338, a drop in pressure in condensation chamber 49, 149, 249, 349 at outlet orifice 35, 135, 235, 335, and maybe by the movement of air into pyrolysization cooking space 38 through an inlet orifice 37, 337, if included and not otherwise blocked by the herb 382 itself or tight fitting parts (e.g., parts 310 and 351). The gasified essential oil condenses into an aerosol cloud 67, 167, 267 after exiting the outlet orifice 35, 135, 235, 335 due to a relatively quick drop in temperature as the gasified products mix with lower temperature gas (e.g., bypass air) outside of mini oven 30, 130, 230, 330. The aerosol cloud 67, 167, 267 of condensed essential oil then is moved to a mouthpiece 65, 165, 265, 365 responsive to suction at the mouthpiece produced by a user 90, which drops the pressure to a vacuum at outlet orifice 35, 135, 235, 335. This sudden drop in pressure can assist in causing quick gasification of the oils in the heated herb 82, 182, 282, 382 by temperarily dropping the boiling point of the essential oil that is function of temperature and pressure. After the original medicinal herb 82, 182, 282, 382 is exhausted, meaning that most or all of its essential oils have been gasified and removed from mini oven 30, 130, 230, 330, the mini oven may be prepared for a subsequent use. This may be done by removing residue of the medicinal herb, which is mostly biochar, from the mini oven 30, 130, 230, 330. The pyrolysizing step may include heating the medicinal herb above 450° Fahrenheit, and limiting production of smoke by starving the mini oven of oxygen. In other words, those skilled in the art will appreciate that smoke is a product of combustion, and one could expect the medicinal herb to combust at temperatures above 450° Fahrenheit if those temperatures occur in air at atmospheric pressure, but pyrolysization according to the present disclosure is not done in air but instead in a carbon dioxide rich gas that may include some air. Non-thermally conductive glass jar 144, 244, 344 isolates the extraction conduit 163, 263, 363 and the mouthpiece 165, 265, 365 from the high temperatures that could be substantially greater than 450 F associated with the heating element 110, 210, 310. Preferably, the gasified essential oil is condensed into an aerosol cloud outside of the mini oven 30, 130, 230, 330 but inside of a window space 66, 166, 266, 366. The aerosol cloud is then moved from the window space 66, 166, 266, 366 toward the mouth piece 65, 165, 265, 365. This may allow a user to adjust a power to the heating element 10, 210, 310 or flame power 200 to heating element 110, 310 positioned in the mini oven 30, 130, 230, 330 or a suction rate at mouth piece 65, 165, 265, 365 responsive to a density of the aerosol cloud 67, 167, 267 visible in the window space 66, 166, 266, 366.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims. Glossary

“Enclosure” means a nearly closed volume that, but for an outlet orifice(s) and an inlet orifice(s), if any, would be a closed volume with no fluid connection between the interior space and an exterior surface of the enclosure.

“Container” means a solid body that defines a nearly closed volume with respect to space outside of the container.

“Orifice” means a passageway that produces a flow restriction to fluid moving between volumes connected by the orifice. In this disclosure, orifices appear as a flow choke point between volumes.

“Biochar” means the blackish carbon rich solid material residue from pyrolysis, which is a thermochemical conversion of biomass in an oxygen-starved environment. Biochar is not ash.

“Closed volume” means a space fluidly isolated from surroundings by a wall that defines the space.

Chamber and space mean a clearly defined fixed volume, which if not a closed volume, is connected to another chamber or space or atmosphere through an orifice

Pyrolysis means something other than moving pre-heated air over medicinal herb at a temperature greater than a gasification temperature of an essential oil of the medicinal herb. Pyrolysis is also different from combustion whose primary solid residue is ash. Pyrolysis is an endothermic process.

Incandescent lamp bulb means a metallic filament that is enclosed by a glass globe and glows responsive to electrical current.

### Unclaimed Subject Matter

A mini oven heating element comprises a incandescent lamp bulb that includes a tungsten coil immersed in a gas different from air and enclosed in a glass globe that defines a cylindrical segment with a length about equal to a diameter of the cylindrical segment, and includes two electrical conductors extending parallel to a centerline of the cylindrical segment and sized to mate with a lamp socket base; and a metallic heat exchanger in contact with, and covering, the cylindrical segment of the glass globe. 2. The mini oven heating element of definition 1 wherein the metallic heat exchanger includes a copper or aluminum tube with an inner diameter that matches the diameter of the cylindrical segment. 3. The mini oven heating element of definition 1 wherein the metallic heat exchanger includes a circular end cover that covers most to all of one end of the glass globe. 4. The mini oven heating element of definition 1 wherein the metallic heat exchanger has a greater thermal mass and a higher thermal conductivity than the glass globe. 5. The mini oven heating element of definition 1 wherein the incandescent lamp bulb is a 12V halogen lamp bulb with a G4 base configuration. 6. A mini oven comprises a mini oven heating element that includes a incandescent lamp bulb in contact with, and at least partially covered by, a metallic heat exchanger; a lamp socket base; an enclosure that defines an interior space, includes an external surface, and defines at least one outlet orifice extending between the interior space and the external surface; wherein the mini oven heating element is positioned in the interior space and electrically connected to the lamp socket base; wherein the mini oven heating element and the enclosure define a pyrolysizing cooking space that is less than all of the interior space and fluidly connected to the external surface through the outlet orifice. 7. The mini oven of definition 6 wherein the pyrolysizing cooking space, with an exception of the outlet orifice, is a closed volume. 8. The mini oven of definition 6 wherein the enclosure includes a cylindrical container with a detachable cover; the lamp socket base is mounted to the cover, and the cylindrical container defines the outlet orifice. 9. The mini oven of definition 6 wherein the lamp socket base is mounted in an externally threaded lamp tube that extends through, and is mounted to, the enclosure. 10. The mini oven of definition 6 wherein the enclosure includes an aluminum reflection surface facing the metallic heat exchanger. 11. The mini oven of definition 6 wherein at least one of the enclosure and the lamp socket base define at least one inlet orifice fluidly connected to the pyrolysizing cooking space. 12. The mini oven of definition 6 wherein the pyrolysizing cooking space has a total volume that is less than 10 ml. 13. The mini oven of definition 6 wherein the enclosure includes a detachable cover threadably attached to a cylindrical container. 14. The mini oven of definition 6 wherein the pyrolysizing cooking space has a cross sectional area at least one order of magnitude greater than a combined total flow area of the at least one outlet orifice. 15. The mini oven of definition 6 wherein the mini oven heating element includes a incandescent lamp bulb that includes a tungsten coil immersed in a gas different from air and enclosed in a glass globe that defines a cylindrical segment with a length about equal to a diameter of the cylindrical segment, and includes two electrical conductors extending parallel to a centerline of the cylindrical segment and sized to fit a lamp socket base; and a metallic heat exchanger in contact with, and covering, the cylindrical segment of the glass globe.

What is claimed is:

1. A method of separating essential oil from a medicinal herb, comprising the steps of:
opening a mini oven;
positioning a personal amount of the medicinal herb in the mini oven;
closing the mini oven;
pyrolysizing the medicinal herb in the mini oven, which includes displacing oxygen from the mini oven with carbon dioxide responsive to decarboxylation of the medicinal herb;
moving gasified essential oil out of the mini oven through an outlet orifice;
condensing the gasified essential oil into an aerosol cloud;
moving the aerosol cloud of condensed essential oil to a mouthpiece; and
preparing the mini oven for a subsequent use by removing residue of the medicinal herb, which is mostly biochar, from the mini oven.

2. The method of claim 1 wherein the pyrolysizing step includes heating the medicinal herb above 450 F; and
limiting production of smoke from combustion by starving the mini oven of oxygen.

3. The method of claim 1 including a step of inducing a pressure differential on opposite sides of the outlet orifice between a condensation chamber and the mini oven responsive to suction at the mouthpiece.

4. The method of claim 1 wherein the condensing step includes condensing the gasified essential oil into the aerosol cloud outside of the mini oven and inside a window space; and
moving the aerosol cloud from the window space toward the mouthpiece.

5. The method of claim 1 including a step of adjusting power to a heating element positioned in the mini oven responsive to a density of the aerosol cloud visible in a window space.

6. A personal essential oil extractor comprising:
a container that defines a condensation chamber;
a mini oven including a heating element positioned within an enclosure;
wherein the heating element and the enclosure define a pyrolysizing cooking space that is fluidly connected to the condensation chamber exclusively through at least one outlet orifice;
an extraction conduit with one end opening into the condensation chamber and an opposite end that opens through a mouthpiece;
wherein gasified compounds from a medicinal herb generated responsive to pyrolysis in the pyrolysizing cooking space move through the outlet orifice, into the condensation chamber toward the extraction conduit responsive to suction at the mouthpiece; and
wherein oxygen in the pyrolysizing cooking space is displaced by carbon dioxide responsive to decarboxylation of medicinal herb positioned in the pyrolysizing cooking space.

7. The essential oil extractor of claim 6 wherein the condensation chamber includes a window space that is visible from outside the container through a window; and
wherein the outlet orifice is fluidly separated from the one end by the window space such that essential oil that is gasified in the mini oven and moved through the outlet orifice condenses to an aerosol cloud visible in the window space through the window by a user of the essential oil extractor.

8. The essential oil extractor of claim 6 wherein the enclosure, cooking space, with an exception of the outlet orifice, is a closed volume.

9. The essential oil extractor of claim 6 wherein the pyrolysizing cooking space is positioned inside the container.

10. The essential oil extractor of claim 6 wherein the condensation chamber of the container, with an exception of a fluid connection to the extraction conduit and the outlet orifice, is a closed volume.

11. The essential oil extractor of claim 6 wherein the enclosure includes an inlet orifice to the pyrolysizing cooking space that has a smaller flow area than a total flow area of the at least one outlet orifice.

12. The essential oil extractor of claim 6 including bypass orifice that opens directly into the condensation chamber and bypasses the enclosure.

13. The essential oil extractor of claim 6 including an analog potentiometer electrically connected to the heating element, and including a manual adjustment knob.

14. The essential oil extractor of claim 6 including a housing covered by a threaded ring and a disc; and
wherein the container is attached to the disc.

15. The essential oil extractor of claim 6 wherein the heating element includes an incandescent lamp bulb in contact with, and at least partially covered by, a metallic heat exchanger;
the mini oven includes a lamp socket base;
wherein the heating element is electrically connected to the lamp socket base; and
wherein the pyrolysizing cooking space has a total volume that is less than 10 ml.

16. The essential oil extractor of claim 6 wherein the enclosure includes an aluminum reflection surface encircling and facing the heating element.

17. The essential oil extractor of claim 6 wherein the container includes a transparent glass jar with a removable lid.

18. The essential oil extractor of claim 6 wherein the pyrolysizing cooking space has a cross sectional area at least one order of magnitude greater than a combined total flow area of the at least one of the outlet orifice.

19. The essential oil extractor of claim 6 wherein the heating element includes a incandescent lamp bulb that includes a tungsten coil immersed in a gas different from air and enclosed in a glass globe that defines a cylindrical segment with a length about equal to a diameter of the cylindrical segment, and includes two electrical conductors extending parallel to a centerline of the cylindrical segment and sized to fit a lamp socket base; and
a metallic heat exchanger in contact with, and covering, the cylindrical segment of the glass globe.

20. A personal essential oil extractor comprising:
means, including a mini oven, for pyrolysizing a personal consumption quantity of a medicinal herb;
means, including an enclosure of the mini oven, for displacing oxygen from the mini oven with carbon dioxide responsive to decarboxylation of the medicinal herb;
means, including an outlet defined by the mini oven, for moving a gasified portion of the medicinal herb generated responsive to the pyrolysization out of the mini oven to a mouthpiece; and
means, including a size of the outlet, for trapping biochar, which is a majority of a residue from the medicinal herb generated responsive to the pyrolysization, in the mini
